(19) United States
(12) Patent Application Publication
(10) Pub. No.: US 2003/0133905 A1
(43) Pub. Date: Jul. 17, 2003

(54) Composition for treating contact lenses in the eye

(76) Inventors: Zhenze Hu, Pittsford, NY (US); Andrea Lever, Pittsford, NY (US); Lisa C. Simpson, Rochester, NY (US)

Correspondence Address: BAUSCH & LOMB INCORPORATED, ONE BAUSCH & LOMB PLACE, ROCHESTER, NY, 14604-2701, US

(21) Appl. No.: 10/314,829

(22) Filed: Dec. 9, 2002

Related U.S. Application Data
(60) Provisional application No. 60/343,086, filed on Dec. 20, 2001.

(51) Int. Cl. A61K 31/785; A61K 31/717; A61K 31/14

(52) U.S. Cl. 424/78.31; 424/78.38; 514/57; 514/642; 424/78.32

(57) ABSTRACT

A method of preventing and/or reducing deposition of denatured proteins on a contact lens while worn on the eye involves distilling in the eye an ophthalmically compatible composition including tromethamine in an amount effective to prevent or reduce protein denaturation. The compositions preferably include a demulcent, and may include conventional eye drop components such as a preservative, a buffering agent, a chelating agent, an osmolality adjusting agent, and/or a surfactant.

Priority is hereby claimed in the present nonprovisional application to Provisional Application Serial No. 60/343,086 filed Dec. 20, 2001, in accordance with 37 CFR 1.78(a)(4).

### FIELD OF THE INVENTION

This invention relates to compositions and methods for treating contact lenses, especially while the contact lens is worn in the eye. The compositions prevent protein denaturation in the eye, thus preventing denatured proteins from accumulating on the contact lens surface, and/or reduces the amount of denatured protein, thus reducing the amount of denatured protein on the contact lens surface.

### BACKGROUND OF THE INVENTION

In the normal course of wearing contact lenses, tear film and debris composed of proteinaceous, oily, sebaceous and related organic matter have a tendency to deposit and build up on lens surfaces. As part of the routine care regimen, contact lenses must be cleaned to remove these tear film deposits and debris. If these deposits are not properly removed, both the wettability and optical clarity of the lenses are substantially reduced causing discomfort for the wearer.

Conventionally, the cleaning of contact lenses is accomplished with one or both of two general classes of cleaners. Surfactant cleaners, generally known as “daily cleaners” because of their recommended daily use, are effective for the removal of most carbohydrate and lipid derived matter. For this daily cleaning regimen, the contact lens is removed from the eye and treated with the surfactant cleaner. However, these cleaners are not as effective for the removal of proteinaceous matter such as lysozyme. Typically, proteolytic enzymes derived from plant, animal, and microbial sources are used to remove the proteinaceous deposits. These enzymatic cleaners are typically recommended for weekly use and are conventionally employed by dissolving enzyme tablets or liquid enzyme formulations in suitable aqueous solutions, where the contact lens is soaked in the solution.

Proteinaceous matter deposited on a contact lens surface mainly includes proteins native to the eye, such as lysozyme, albumin and mucin. One of the reasons proteinaceous matter deposited on a contact lens is more difficult to remove is that the proteins typically denature once they accumulate on the contact lens surface; the denaturation allows a greater hydrophobic interaction with the hydrophilic contact lens surface. In other words, denatured proteins are more difficult to remove from a contact lens surface than native proteins. Additionally, whereas proteins native to the eye typically do not irritate the eye, denatured proteins on a contact lens surface tend to reduce comfort.

The present invention recognizes that it would be advantageous to prevent or reduce build-up of denatured proteins on a contact lens while worn by the contact lens wearer. The contact lens would be more comfortable to wear for a longer period of time. Additionally, when the contact lens is ultimately removed from the eye, the lens would be easier to clean, i.e., easier to remove any protein deposited on the lens surface.

Various solutions for dispensing directly in the eye while a contact lens is worn are known. As a first example, solutions commonly referred to as “rewetting drops” are used to enhance wettability of the lens surface without removing the lens from the eye, for example, by forming a hydrophilic film on a lens surface. In other words, these rewetting drops facilitate formation of a film over the contact lens. In some cases, a coating is formed for which proteins have little affinity and provides a prophylactic effect. An example, U.S. Pat. No. 5,209,865 (Winterton et al.) discloses a composition including particular classes of poloxamine and poloxamer surfactants that forms a hydrophilic film on the lens surface for which proteins and lipids have little affinity.

As another example, U.S. Pat. No. 6,037,328 (Hu et al.) discloses compositions suitable for application to the eye of a contact lens wearer, comprising an ethyoxylated glucose derivative, tyloxapol, and a poloxamer or polyoxamine surfactant. These compositions are described as useful for both cleaning and rewetting the lens surface.

As yet another example, U.S. Pat. No. 6,096,138 (Heiler et al.) discloses compositions including a moderately charged polyquaternium polymer that may be used as in-the-eye or out-of-eye inhibitors of proteinaceous deposits on hydrophilic contact lenses, where the polyquaternium polymer inhibits the deposition of protein on contact lenses.

Additionally, U.S. Pat. No. 6,274,133 (Hu et al.) discloses a composition for treating contact lenses while worn in the eye and including a cationic cellulose polymer that binds to the lens and prevents the accumulation of lipids, proteins and other materials on the lens. The compositions are especially useful for silicone hydrogel contact lenses intended for extended wear, i.e., contact lenses left in the eye overnight and especially for an extended period of at least seven days.

U.S. Pat. No. 5,422,073 (Mowrey-McKee et al.) discloses compositions for disinfecting contact lens containing tromethamine in an amount of 0.6 to 2 weight percent, where tromethamine has a synergistic microbicidal effect when employed with other antimicrobial agents such as polyhexamethylene biguanide (PHMB).

### SUMMARY OF THE INVENTION

According to a first embodiment, this invention provides a method of preventing deposition of denatured proteins on a contact lens while worn on the eye. The method comprises dispensing in the eye an aqueous composition that comprises tromethamine in an amount effective to prevent denaturation of proteins in the eye. Accordingly, denatured proteins, which are more difficult to remove once bound to a contact lens surface, are not present to bind to the contact lens surface.

According to a second embodiment, this invention provides a method of reducing deposition of denatured proteins on a contact lens while worn on the eye. This method comprises dispensing in the eye an aqueous composition that comprises tromethamine in an amount effective to reduce the amount of denatured protein accumulated on the contact lens. Additionally, even if denatured proteins have already accumulated on the contact lens surface, the composition may convert partially denatured protein back to its native state, thus rendering it easier to remove from the contact lens surface.

According to other embodiments, the invention provides a method that comprises dispensing an aqueous composition in the eye of a contact lens wearer while a contact lens is worn on the eye, where the composition comprises tromethamine in an amount effective to prevent or reduce protein denaturation on the contact lens, and a demulcent. The demulcent may include a non-polymeric demulcent, and/or a polymeric demulcent. The composition may further include a preservative, a buffering agent, a chelating agent, an osmolality adjusting agent, and/or a surfactant.

### DETAILED DESCRIPTION OF THE INVENTION

The present invention may be used with all contact lenses such as conventional hard, soft, rigid and soft gas permeable, and silicone (including both hydrogel and non-hydrogel) lenses, but is preferably employed with soft hydrogel lenses. Such lenses are commonly prepared from hydrophilic monomers such as 2-hydroxyethyl(meth)acrylate, N-vinylpyrrolidone, glycerol(meth)acrylate, and (meth)acrylic acid. In the case of silicone hydrogel lenses, a silicone-containing monomer is copolymerized with at least one hydrophilic monomer. Such lenses absorb significant amounts of water, typically from 10 to 80 percent and more typically 20 to 70 percent water by weight. The compositions and methods of this invention are especially useful for silicone hydrogel contact lenses, since such contact lenses are specifically designed for extended wearing times, for example, one week or up to thirty days without being removed from the wearer's eye. In other words, the compositions prevent build-up of denatured proteins on the contact lens surface, and reduce the amount of denatured proteins already bound to the lens surface, thus facilitating longer wear regimens of contact lenses without removing the lens for cleaning.

The compositions employed in this invention are aqueous solutions. The compositions include, as an essential component, 2-amino-2-hydroxymethyl-1,3-propanediol, also known by the names tris(hydroxymethyl)aminomethane, tromethamine and TRIS. This component is known as a buffer for contact lens solutions and is commercially available. In the present solutions, tromethamine is employed in amount effective to prevent or reduce denaturation of proteins, preferably at least 0.05 weight percent, more preferably 0.05 to 1 weight percent, and most preferably 0.1 to 0.5 weight percent. Tromethamine is commercially available, for example under the tradename Tris Amino® (Angus Chemical Company, Northbrook, Ill.).

In addition to water and tromethamine, according to preferred embodiments the subject compositions include at least one demulcent. As used herein the term demulcent is intended to mean an agent, usually a water-soluble polymer, which protects and lubricates mucous membrane surfaces of the eye and relieves dryness and irritation. Within this meaning, the terms “humectant” and “wetting agent” are also commonly used to describe these materials.

A first class of suitable demulcents are non-polymeric demulcents. Examples include glycerin, propylene glycol, and other non-polymeric diols and glycols.

A second class of demulcents are polymer demulcents. Examples include: polyvinyl alcohol (PVA), polyvinyl pyrrolidone (PVP), cellulose derivatives and polyethylene glycol. Cellulose derivatives and PVA may be used to also increase viscosity of the composition, and offer this advantage if desired. Specific cellulose derivatives include: hydroxypropyl methyl cellulose, carboxymethyl cellulose, methyl cellulose, hydroxyethyl cellulose, and cationic cellulose derivatives. As disclosed in U.S. Pat. No. 6,274,133, cationic cellulosic polymers also help prevent accumulation of lipids and proteins on hydrophilic lens surfaces. Examples of such polymers include commercially available water soluble polymers available under the CTFA (Cosmetic, Toiletry, and Fragrance Association) designation Polyquaternium-10, including the cationic cellulosic polymers available under the tradename UCARE®) Polymer (Amerchol Corp., Edison, N.J.). Generally, these cationic cellulose polymers contain quaternized N,N-dimethyl amino groups along the cellulosic polymer chain.

The demulcents used in the present invention are used in an effective demulcifying amount, i.e., an amount effective to sufficiently lubricate mucous membrane surfaces and to relieve dryness and irritation and/or an amount effective to wet a contact lens surface. The specific quantities of demulcents used in the present invention will vary depending upon the application. However, the demulcents will typically be included in an amount from about 0.01 to about 5 weight percent to preferably from about 0.1 to about 2 weight percent. Also, generally, the non-polymeric demulcents will be used in higher amounts than the polymeric demulcents. When the compositions include both non-polymeric and polymeric demulcents, the non-polymeric demulcents are preferably included in an amount from about 0.1 to about 5 weight percent, and the polymeric demulcents are preferably included in an amount from about 0.01 to about 2 weight percent.

Additionally, the compositions may also contain various other components including, but not limited to preservative agents, chelating and/or sequestering agents, osmolality adjusting agents, and surfactants.

The composition may include at least one preservative agent. As used herein, the term preservative agent is intended to mean a non-oxidative antimicrobial agent which derives its antimicrobial activity through a chemical or physicochemical interaction with organisms, employed in an amount effective to prevent microbial growth during storage of the composition in case the composition is contacted with microbes during storage. Additionally, since the compositions are instilled directly in the eye while a contact lens is worn, the antimicrobial agent needs to be an ophthalmically acceptable antimicrobial agent.

Suitable antimicrobial agents include quaternary ammonium salts, which do not include significant hydrophobic portions, e.g. alkyl chains comprising more than six carbon atoms. Examples of suitable quaternary ammonium salts for use in the present invention include poly[(dimethyliminio)-2-butene-1,4-diyl chloride] and [4-tris(2-hydroxyethyl) ammonio]-2-butenyl-w-[tris(2-hydroxyethyl)ammonio] dichloride (chemical registry no. 75345-27-6) generally available as Polyquaternium 1 (ONYX Scientific Limited, Sunderland, United Kingdom); biguanides and their salts, such as alexidine and polyhexamethylene biguanides such as PHMB available under the tradename Cosmocil™ Co (ICI Americas, Inc., Wilmington Del.); benzalkonium chloride (BAK); and sorbic acid.

It is noted that it is not necessary for the compositions to include the preservative agent. In other words, the composition may be “preservative-free”. In such a case, the composition should be packaged and stored in a container that is designed to prevent microbial contamination of its contents. Containers so designed are commonly referred to as “preservative-free containers”, including single-dose containers.

Since the subject compositions are designed for in-eye use, it is not necessary that an antimicrobial agent be present in an amount effective for disinfecting a contact lens, as in conventional lens soaking and disinfecting solutions. However, if it is desired that the subject compositions have the dual purpose of an in-eye drop and a lens soaking solution, then an antimicrobial agent would need to be present in a disinfecting amount. Preferably, a disinfecting amount is that which will reduce the microbial burden by a certain number of log orders within a certain period of time, depending on the particular microorganism involved. Most preferably, a disinfecting amount is an amount which will eliminate the microbial burden on a contact lens when used in regimen for the recommended soaking time (FDA Chemical Disinfection Efficacy Test—July, 1985 Contact Lens Solution Draft Guidelines).

As mentioned, the subject composition may include at least one chelating agent, also referred to as a sequestering agent, especially when the composition includes a preservative agent. Chelating agents bind heavy metal ions, which might otherwise react with the lens and/or protein deposits and collect on the lens. Chelating agents are well known in the art, and examples of preferred chelating agents include ethylenediaminetetraacetic acid (EDTA) and its salts, especially disodium EDTA. Such agents are normally employed in amounts from about 0.01 to about 2.0 weight percent, more preferably from about 0.01 to about 0.3 weight percent. Other suitable sequestering agents include gluconic acid, citric acid, tartaric acid and their salts, e.g. sodium salts.

The subject composition may be designed for a variety of osmolalities, but it is preferred that the composition is iso-osmal with respect to eye fluids. Specifically, it is preferred that the composition has an osmotic value of less than about 350 mOsm/kg, more preferably from about 175 to about 330 mOsm/kg, and most preferably from about 280 to about 320 mOsm/Kg. At least one osmolality adjusting agent may be employed in the composition to obtain the desired final osmolality. Examples of suitable osmolality adjusting agents include, but are not limited to, sodium and potassium chloride, monosaccharides such as dextrose, calcium and magnesium chloride, and low molecular weight polyols such as glycerin and propylene glycol. Typically, these osmolality adjusting agents are used individually in amounts ranging from about 0.01 to 5 weight percent and preferably, from about 0.1 to about 2 weight percent.

The subject composition has an ophthalmically compatible pH, which generally will range between about 6 to about 8, and more preferably between 6.5 to 7.8, and most preferably about 7 to 7.5. Conventional buffers may be employed to obtain the desired pH value. As mentioned, tromethamine is known as a buffer for contact lens treating compositions. However, the subject compositions may include a supplemental buffering agent. In other words, the composition may include a “mixed buffer” of tromethamine and one or more supplemental buffer agents. Suitable buffers include for example borate buffers based on boric acid and/or sodium borate, phosphate buffers based on Na₂HPO₄, NaH₂PO₄ and/or KH₂PO₄, a citrate buffer based on potassium citrate and/or citric acid, sodium bicarbonate, and combinations thereof. Generally, buffers will be used in amounts ranging from about 0.05 to 2.5 weight percent, and preferably, from 0.1 to 1.5 weight percent.

It will be understood that some components of the present composition possess more than one functional attribute. For example, as mentioned, tromethamine provides the effect of preventing protein denaturation, but also contributes a buffering effect. Cellulose derivatives are suitable polymeric demulcents, but are also referred to as “viscosity increasing agents” to increase viscosity of the composition if desired. Glycerin is a suitable non-polymeric demulcent but may also contribute to adjusting tonicity.

The composition of the present invention may include at least one ophthalmically acceptable surfactant, which may be cationic, anionic, nonionic or amphoteric. Preferred surfactants are amphoteric or nonionic surfactants. The surfactant should be soluble in the aqueous solution and non-irritating to eye tissues.

Many nonionic surfactants comprise one or more chains or polymeric components having oxyalkylene (—O—R—) repeats units wherein R has 2 to 6 carbon atoms. Representative non-ionic surfactants comprise block polymers of two or more different kinds of oxyalkylene repeat units, which ratio of different repeat units determines the HLB of the surfactant. For example, poloxamers are polyoxyethylene, polyoxypropylene block polymers and available under the tradename Pluronic™ (BASF Wyandotte Corp., Wyandotte, Mich.). Poloxamines are ethylene diamine adducts of such polyoxyethylene, polyoxypropylene block polymers available under the tradename Tetronic™ (BASF Wyandotte Corp.), including poloxamine 1107 (Tetronic 1107) having a molecular weight from about 7,500 to about 27,000 wherein at least 40 weight percent of said adduct is poly(oxyethylene). Other suitable non-ionic surfactants include for example polyethylene glycol esters of fatty acids, e.g. coconut, polysorbate, polyoxyethylene or polyoxypropylene ethers of higher alkanes (C₁₂-C₁₈), polysorbate 20 available under the trademark Tween® 20 (Sigma Aldrich Company, St. Louis, Mo.), polyoxyethylene (23) lauryl ether available under the tradename Brij® 35 (Sigma Aldrich Company), polyoxyethyene (40) stearate available under the tradename Myrj® 52 (Sigma Aldrich Company) and polyoxyethylene (25) propylene glycol stearate available under the tradename Atlas®) G 2612 (Sigma Aldrich Company).

Another useful class of surfactants are the hydroxyalkylphosphonates, such as those disclosed in U.S. Pat. No. 5,858,937 (Richards et al.), and available under the tradename Dequest® (Montsanto Co., St. Louis, Mo.).

Amphoteric surfactants suitable for use in compositions according to the present invention include materials of the type offered commercially under the tradename Miranol™ (Rhodia HPCII, Cranbury, N.J.). Another useful class of amphoteric surfactants is exemplified by cocoamidopropyl betaine, commercially available from various sources.

Various other ionic as well as amphoteric and anionic surfactants suitable for in the invention can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

Preferably, the surfactants, when present, are employed in a total amount from about 0.01 to about 15 weight percent, preferably 0.1 to 5.0 weight percent, and most preferably 0.1 to 1.5 weight percent.

As an illustration of the present invention, several examples are provided below. These examples serve only to further illustrate aspects of the present invention and should not be construed as limiting the invention.

### EXAMPLE 1

A series of 10-mi test solutions, listed in the following table, were prepared. Each solution included saline and 20 mM of buffering agent as identified in Table 1 below. To each test solution was added 1 mg/ml of hen egg lysozyme as well as a phosphate buffered saline (PBS) control. The test solutions were mixed slowly with a stir bar until the lysozyme was incorporated into the solutions. Five ml of each lysozyme-containing test solution was retained as the unheated control. The remaining 5 ml of each lysozyme-containing test solution were placed in glass lens vials, capped with silicone stoppers and incubated in a shaking water bath at 80° C., 40 revolutions per minute (rpm) for 1 hour. These heating conditions are sufficient to denature the lysozyme, absent a stabilization effect provided by the buffering agents.

The vials were allowed to come to ambient temperature before testing. A 0.00025g/ml suspension of M. luteus was prepared from lyophilized cells in PBS. The suspension was continually mixed on a stir plate during the testing period to prevent the suspension from settling.

For each set of test solutions, the following were tested (sample): a heated lysozyme-containing test solution (“lysozyme+heat”); an unheated lysozyme-containing test solution (“lysozyme/no heat”); and a test solution without lysozyme (“no lysozyme”). One ml of each sample was placed into a glass test tube to which 9 ml of M. luteus suspension was added and vortexed. A 1-ml sub-sample was placed into a disposable cuvette and evaluated on a UV-vis spectrophotometer at 450 nm. This procedure was performed for each sample at 0, 5 and 10 minutes. Each of the solutions was evaluated in triplicate. Each of the three optical density measurements from the triplicate samples was averaged. The resulting mean value for the 5 and 10 minute time points was used to determine the Percentage Change at the 5 and 10 minute time points.

As can be seen in the table, the compositions containing tromethamine were generally more effective at stabilizing the protein against denaturation. Thus, these compositions are expected to reduce the amount of denatured protein available to bind to a contact lens surface, noting that native protein is removed from a contact lens relatively easily and not particularly irritating to the eye, whereas denatured protein is opaque, adheres tenaciously to a contact lens surface, and leads to eye irritation and discomfort.

TABLE 1

| | | Time | | | Percent Change | |
|---|---|---|---|---|---|---|
| Solution | Treatment | 0 Min | 5 Min | 10 Min | 5 Min | 10 Min |
| Borate | lysozyme/no heat | 0.619 | 0.053 | 0.034 | 91.44 | 94.51 |
| | lysozyme + heat | 0.872 | 0.681 | 0.390 | 21.90 | 55.28 |
| | no lysozyme | 0.853 | 0.853 | 0.854 | 0.00 | −0.12 |
| Phosphate | lysozyme/no heat | 0.634 | 0.052 | 0.029 | 91.80 | 95.43 |
| | lysozyme + heat | 0.861 | 0.858 | 0.852 | 0.34 | 1.05 |
| | no lysozyme | 0.856 | 0.852 | 0.854 | 0.47 | 0.23 |
| Tris | lysozyme/no heat | 0.654 | 0.048 | 0.028 | 92.66 | 95.72 |
| | lysozyme + heat | 0.810 | 0.154 | 0.117 | 80.99 | 85.56 |
| | no lysozyme | 0.859 | 0.854 | 0.854 | 0.58 | 0.58 |
| Dequest | lysozyme/no heat | 0.629 | 0.051 | 0.032 | 91.89 | 94.91 |
| | lysozyme + heat | 0.857 | 0.848 | 0.842 | 1.05 | 1.75 |
| | no lysozyme | 0.852 | 0.850 | 0.848 | 0.23 | 0.47 |
| Citrate | lysozyme/no heat | 0.654 | 0.049 | 0.030 | 92.51 | 95.41 |
| | lysozyme + heat | 0.877 | 0.851 | 0.785 | 2.96 | 10.49 |
| | no lysozyme | 0.857 | 0.850 | 0.850 | 0.82 | 0.82 |
| Citrate + | lysozyme/no heat | 0.611 | 0.057 | 0.037 | 90.67 | 93.94 |
| Phosphate | lysozyme + heat | 0.864 | 0.839 | 0.827 | 2.89 | 4.28 |
| | no lysozyme | 0.852 | 0.849 | 0.856 | 0.35 | −0.47 |
| Citrate + | lysozyme/no heat | 0.602 | 0.050 | 0.033 | 91.69 | 94.52 |
| Borate | lysozyme + heat | 0.854 | 0.817 | 0.785 | 4.33 | 8.08 |
| | no lysozyme | 0.848 | 0.844 | 0.846 | 0.47 | 0.24 |
| Borate + | lysozyme/no heat | 0.564 | 0.051 | 0.036 | 90.96 | 93.62 |
| Tris | lysozyme + heat | 0.836 | 0.216 | 0.167 | 74.16 | 80.02 |
| | no lysozyme | 0.847 | 0.841 | 0.843 | 0.71 | 0.47 |
| Phosphate + | lysozyme/no heat | 0.598 | 0.050 | 0.031 | 91.64 | 94.82 |
| Borate | lysozyme + heat | 0.847 | 0.841 | 0.838 | 0.71 | 1.06 |
| | no lysozyme | 0.848 | 0.852 | 0.847 | −0.47 | 0.12 |
| Tris + | lysozyme/no heat | 0.575 | 0.048 | 0.030 | 91.65 | 94.78 |
| Dequest | lysozyme + heat | 0.846 | 0.605 | 0.349 | 29.98 | 59.61 |
| | no lysozyme | 0.830 | 0.843 | 0.843 | −1.57 | −1.57 |
| M. Luteus + | no lysozyme | 0.836 | 0.849 | 0.838 | −1.56 | −0.24 |
| PBS—Control | | | | | | |

### EXAMPLES 2 AND 3

The following are representative compositions of the present invention. The subject compositions identified as Example 2 and Example 3 in Table 2 below, were prepared according to the following method. The non-polymeric components—such as sodium borate, tromethamine (Tris), sodium chloride, glycerin, EDTA and sorbic acid—are added sequentially to a volume of heat water (about 50° C.) that amounts to about 70-85% of the final batch volume. This addition is done under constant agitation, and each component is allowed to dissolve or disperse before adding the next component. Subsequently, the polymeric components—such as the cationic cellulose polymer, poloxamer, and poloxamine—are added sequentially under agitation, ensuring adequate dispersion of each polymer. The resulting solution is mixed until complete dissolution is achieved. The batch is cooled under agitation to room temperature. The pH is adjusted to about 7.0 by incrementally adding 1 N NaOH or 1 N HCl, and then the final volume is achieved by adding water (at 20-30° C.) and mixing for at least 15 minutes.

TABLE 2

| Ingredients (w/w %) | Example 2 | Example 3 |
|---|---|---|
| Sodium Borate | 0.134 | 0.134 |
| Triethanolamine | 0.121 | 0.121 |
| Glycerin | 1 | 1 |
| Tetronic 1107 | 1 | 1 |
| Pluronic F127 | 2 | 2 |
| EDTA | 0.05 | 0.05 |
| NaCl | 0.38 | 0.38 |
| Sorbic Acid | 0.165 | 0.165 |
| UCARE ® Polymer | — | 0.02 |

| | |
|---|---|
| Purified water | q.s to 100 |

Compositions identified as Example 2 and Example 3 in Table 2 were tested according to the procedure described in the Example 1. As shown by the test data set forth below in the Table 3, the lens drop solutions containing tromethamine were very effective at stabilizing the protein against denaturation.

TABLE 3

| | | Time | | | Percent Change | |
|---|---|---|---|---|---|---|
| Solution | Treatment | 0 Min | 5 Min | 10 Min | 5 Min | 10 Min |
| Example 2 | lysozyme/no heat | 0.798 | 0.076 | 0.055 | 92.42 | 94.51 |
| | lysozyme + heat | 0.942 | 0.146 | 0.088 | 85.47 | 91.29 |
| | no lysozyme | 1.007 | 1.008 | 1.005 | −0.07 | 0.17 |
| Example 3 | lysozyme/no heat | 0.789 | 0.125 | 0.100 | 87.67 | 90.08 |
| | lysozyme + heat | 0.942 | 0.185 | 0.146 | 81.74 | 85.56 |
| | no lysozyme | 1.011 | 1.014 | 1.014 | −0.23 | −0.26 |
| M. Luteus + | no lysozyme | 0.978 | 0.974 | 0.973 | 0.44 | 0.55 |
| PBS—Control | | | | | | |

### EXAMPLE 4

Compositions identified as Example 2 and Example 3 in Table 2 were further evaluated for protein deposition prevention ability against phosphate buffer using image analysis. The results of the evaluation are set forth below in Table 4. For each composition tested, five Group I lenses and five Group IV lenses were prepared in accordance with the following method.

The lenses were removed from their packaging and placed in a beaker of PBS where they remained for approximately 5 minutes. The lenses were then removed and blotted to remove any excess liquid and placed in the test composition for approximately 2 minutes. The lenses were then blotted and placed into glass lens vials with 5 ml of 1 mg/ml lysozyme in PBS and closed with silicone stoppers. The test lenses were deposited in accordance with the following method. Group IV lenses were processed in a water bath at 40 revolutions per minute (rpm), 37° C. for 1 hour. The lenses remained in the bath at 40 rpm while the temperature ramped up to 80° C. (approximately 23 minutes). The lenses remained at 80° C. at 40 rpm for an additional 20 minutes after which they were removed and allowed to come to room temperature. Group I lenses were processed in a water bath at 40 rpm, 80° C. for 20 minutes. The lenses were removed and allowed to come to room temperature. The lenses were removed from the vials, rubbed and rinsed with PBS to remove any loosely deposited protein. The deposited Group I and Group IV lenses were then placed in “flat packs” with PBS and refrigerated until image analysis could be performed. Image analysis was performed on each lens and the image stored. The mean value and standard deviation was recorded. The following calculations were performed:

Difference from Positive Control (PC)=Test (TG) Group Mean−PC Group Mean;

Difference of Test Deposit (TD) from Negative Control (NC)=255*−Difference from PC;

Difference of PC from NC=255*−PC Group Mean; and

% of Deposit Prevented=1−(Difference of TD from NC/Difference of PC from NC)×100. *Note: 255 is taken as the image analysis result for a clean lens without deposits.

The results summarized in the Table 4 below show that both test compositions exhibited a higher percentage of lysozyme deposit prevention with both Group I and Group IV lenses than did the PBS.

TABLE 4

| | | Percent of Deposit |
|---|---|---|
| Formulation | Lens | Prevention |
| Example 2 | ClearView | 29.09 |
| | AcuVue | 67.84 |
| Example 3 | ClearView | 36.01 |
| | AcuVue | 81.71 |
| PBS | ClearView | 0 |
| | AcuVue | 0 |

Although various preferred embodiments have been illustrated, many other modifications and variations of the present invention are possible to the skilled practitioner. It is therefore understood that, within the scope of the claims, the present invention can be practiced other than as herein specifically described.

We claim:

1. A method comprising dispensing an aqueous composition in the eye of a contact lens wearer while a contact lens is worn on the eye, said composition comprising:
tromethamine in an amount effective to prevent or reduce protein denaturation on the contact lens; and a demulcent.

2. The method of claim 1, wherein the composition further comprises at least one member selected from the group consisting of a preservative, a buffering agent, a chelating agent, an osmolality adjusting agent, and a surfactant.

3. The method of claim 1, wherein the composition comprises a non-polymeric demulcent.

4. The method of claim 3, wherein the composition comprises at least one member selected from the group consisting of glycerin and propylene glycol.

5. The method of claim 1, wherein the composition comprises a polymeric demulcent.

6. The method of claim 3, wherein the composition comprises at least one member selected from the group consisting of polyvinyl alcohol, polyvinyl pyrrolidone, a cellulose derivative and polyethylene glycol.

7. The method of claim 6, wherein the composition comprises a cationic cellulose polymer.

8. The method of claim 1, wherein the composition comprises a non-polymeric demulcent and a cellulose derivative.

9. The method of claim 8, wherein the cellulose derivative includes a cationic cellulose polymer.

10. The method of claim 1, wherein the composition comprises:
at least 0.05 weight percent of tromethamine; and
0.01 to about 5 weight percent of a demulcent.

11. The method of claim 1, wherein the composition comprises:
at least 0.05 weight percent of tromethamine;
0.1 to 5 weight percent of at least one non-polymeric demulcent;
0.01 to 2 weight percent of a chelating agent; and
a buffering agent selected from the group consisting borate buffers, phosphate buffers and citrate buffers.

12. The method of claim 11, wherein the composition further comprises a preservative.

13. The method of claim 11, wherein the composition further comprises at least one member selected from the group consisting of poloxamer and poloxamine surfactants.

14. The method of claim 13, wherein the composition comprises 0.01 to 2 weight percent of a cationic cellulose polymer.

15. The method of claim 1, wherein the composition is distilled in the form of drops.

16. A method of preventing deposition of denatured proteins on a contact lens while worn on the eye, said method comprising dispensing in the eye an aqueous composition that comprises tromethamine in an amount effective to prevent denaturation of proteins in the eye.

17. A method of reducing deposition of denatured proteins on a contact lens while worn on the eye, said method comprising dispensing in the eye an aqueous composition that comprises tromethamine in an amount effective to reduce the amount of denatured protein denaturation on the contact lens.

18. A composition comprising tromethamine in an amount effective to prevent or reduce protein denaturation on the contact lens and a demulcent, said composition having a pH and osmolality suitable for instilling the composition in the eye.
